(19) United States
(12) Patent Application Publication
(10) Pub. No.: US 2019/0343858 A1
(43) Pub. Date: Nov. 14, 2019

(54) METHOD OF REGULATING THE CIRCADIAN RHYTHM OF AN INDIVIDUAL COMPRISING ADMINISTERING AN EFFECTIVE AMOUNT OF A CIRCADIAN RHYTHM REGULATOR CONTAINING 1,2-DI-O-GALLOYL-4,6-O-(S)-HEXAHYDROXYDIPHENOYL-B-D-GLUCOSE

(71) Applicant: ORYZA OIL & FAT CHEMICAL CO., LTD,, Aichi (JP)

(72) Inventors: Hiroshi SHIMODA, Aichi (JP); Marina KOHATSU, Aichi (JP); Kazuya TODA, Aichi (JP); Hiromichi MURAI, Aichi (JP)

(21) Appl. No.: 15/762,210

(22) PCT Filed: Jan. 30, 2017
(86) PCT No.: PCT/JP2017/003246

§ 371 Date: Mar. 22, 2018

(51) Int. Cl. A61K 31/7034; A61K 8/60; A61K 9/00; A61K 8/9789; A61K 36/82; A61K 9/68

(52) U.S. Cl. CPC A61K 31/7034; A61K 8/60; A61K 9/0056; A61K 9/0058; A61K 9/0014; A61K 36/82; A61K 8/9789

(57) ABSTRACT

This invention provides a new application to 1,2-di-O-galloyl-4,6-O—(S)-hexahydroxydiphenoyl-ß-D-glucose (i.e. GHG) that is contained in Kenyan purple tea (scientific name: Camellia sinensis; variety name: TRFK306), as well as a new circadian-rhythm regulator that can be widely used in medicines, foods and drinks, and cosmetics. The circadian-rhythm regulator of this invention is characterized in containing the GHG as an active ingredient for promoting the BMAL1 gene-expression. Also, the circadian-rhythm regulator of this invention is characterized in containing the GHG-content extract derived from Kenyan purple tea as an active ingredient for promoting the BMAL1 gene-expression.

### TECHNICAL FIELD

This invention relates to a circadian-rhythm regulator derived from plants, which are applicable to a wide range of fields such as drugs and medicines, foods, drinks, cosmetics or the like.

### TECHNICAL BACKGROUND

Almost all living organisms including man have a circadian rhythm repeating in about a 24-hour cycle of which a biological clock controls physiological activity such as sleep, awakening, hormonal secretion, blood pressure, body temperature or the like. It is pointed out that abnormality of the circadian rhythm causes jet lag, sleep disorder or skin disorder, or it could be a factor in causing lifestyle-related diseases such as obesity, diabetes, high-blood pressure or the like.

A clock gene that expresses a clock protein, and another gene that expresses a protein as its transcription factor, are involved in the molecular mechanism of the circadian rhythm. In the case of mammals, as the clock gene, a gene-cluster such as Per (Period) or the like is identified, and as a gene regarding the transcription factor, a gene-cluster such as BMAL1 or Clock or the like is identified. These gene clusters mutually express one another, thus generating self-sustaining amplitude of nearly 24 hours.

Of the molecular mechanism generating such self-sustaining amplitude, the protein (BMAL1), which is synthetically made by expressing the BMAL1 gene, and the protein (Clock), which is synthetically made by expressing the clock gene, are paired up so as to make up the transcription factor. This transcription factor is combined with the clock gene in promoting its expression, thus synthesizing the clock protein (Per).

The clock protein (Per) has the function of controlling the transcription factor. If the transcription factor is controlled by such a function, the expression of the clock gene decreases as time proceeds. Then, the amount of the clock protein (Per) decreases. If the amount of the clock protein (Per) decreases, the transcription factor will again promote the expression of the clock gene as time proceeds. Then, the amount of the clock protein will increase. This allows the function of the transcription factor again to be controlled by the clock protein.

As such, the two gene clusters form a transcription-translation feedback loop (TTFL), beats out a circadian rhythm of 24 hours.

The main tissue for adjusting the circadian rhythm exists in the suprachiasmatic nucleus (SCN) that is located in the hypothalamus of the brain (central tissue). The clock gene exists not only in the suprachiasmatic nucleus (SCN) as a central tissue, but it exists in most cells of the human body. It is known that the peripheral tissue is expressed with the circadian rhythm. It is also known that in the suprachiasmatic nucleus, the expression-level of the Per gene is outstanding in the daytime, and that the expression-level of the BMAL1 gene is centrally outstanding at night in synchronizing with the above feedback loop. The expression-level of the clock gene, as well as the Per gene, reaches the peak in the daytime. However, that rhythm is not so strong. Thus, it is believed that the BMAL1 gene is the one that controls substantially the rhythm of the Per gene.

Recently, to improve the various symptoms caused by such a variation of the circadian rhythm, R & D for controlling the expression of the clock gene is conducted, and some results are reported such that active substances that can adjust the circadian rhythm is extracted from highly safe plants that are already being used as ingredients in medicines and foods.

For example, Patent Document 1 discloses an activator for expressing the BMAL1 gene, which contains a prune extract or the like as the active substance. Patent Document 2 discloses an adjustor for expressing the BMAL1 gene, which contains an extract of Ganoderma lucidum as the active substance.

### PRIOR ART DOCUMENTS

### Patent Documents

Patent Document 1: Japanese Published Unexamined Patent Application No. 2013-56866
Patent Document 2: Japanese Published Unexamined Patent Application No. 2016-56140
Patent Document 3: International Publication No. 2015-022909

### Non-Patent Document

Non-patent Document 1: Chatterjee et al., Journal of Cell Science 126, 2213-2224(2013)

### DISCLOSURE OF THE INVENTION

### Problems to be Solved by the Invention

On the other hand, as part of the development of bioactive components derived from plants, the inventors of this invention have been providing for research in 1,2-di-O-galloyl-4,6-O—(S)-hexahydroxydiphenoyl-ß-D-glucose (hereinafter referred to as GHG) that is a specific component contained in purple tea originating in Kenya (scientific name: Camellia sinensis; variety name: TRFK306; hereinafter referred to simply as Kenyan purple tea).

The GHG content of plants that are commonly known is less, containing only a few contents of the GHG component. If such a plant contains a high concentration of GHG, it is wild species or the like, which is inappropriate to grow or difficult to obtain. However, in their research, the inventors of this invention identified that a plentiful amount of GHG is contained in Kenyan purple tea, thus making it comparatively easy to obtain a high level of the GHG component.

GHG bioactivity includes an anti-oxidant activity (DPPH radical-scavenging activity and a SOD-like activity); a tyrosinase-inhibitory activity in the conventional manner; as well as a fat-absorption inhibitory activity and a body-weight-increase inhibitory activity that are newly reported in the research result by the inventors of this invention (see Patent Document 3).

As disclosed above, the inventors of this invention, in their research of the bioactivity of GHG derived from Kenyan purple tea, focused their research on a biological clock deeply involved with the lifestyle habit and came to study how GHG works on the clock gene. As result of their earnest research, they came to know that GHG affects the BMAL1 gene-expression level in the myoblast cell, and then they achieved the completion of this invention.

This invention provides a new application to GHG that is contained in Kenyan purple tea, as well as a new circadian-rhythm adjustor that can be widely used as medicines, foods and drinks, and cosmetics.

### Means for Solving the Problems

Technical features of this invention for solving the above-referenced problems are as follows.

1. A circadian-rhythm regulator containing GHG as the active ingredient.

2. A circadian-rhythm regulator having an extract containing GHG as the active ingredient, derived from Kenyan purple tea.

3. Food component for regulating the circadian rhythm, containing GHG as the active ingredient.

4. Drink component for regulating the circadian rhythm, containing GHG as the active ingredient.

5. Cosmetics component for regulating the circadian rhythm, containing GHG as the active ingredient.

6. The BMAL1 gene-expression promoter, containing GHG as the active ingredient.

7. The BMAL1 gene-expression promoter having an extract containing GHG derived from Kenyan purple tea.

8. Food component for promoting the BMAL1 gene-expression, containing GHG as the active ingredient.

9. Drinks component for promoting the BMAL1 gene-expression, containing GHG as the active ingredient.

10. Cosmetics component for promoting the BMAL1 gene-expression, containing GHG as the active ingredient.

### Effects of the Invention

This invention allows for the GHG that is obtained from plants, such as Kenyan purple tea or the like, to be used as a new application, i.e. as the circadian-rhythm regulator and BMAL1 gene-expression promoter. It is possible to obtain the active substance GHG efficiently from Kenyan purple tea that is appropriate to grow, thus making it possible to apply the circadian-rhythm regulator and the BMAL1 gene-expression promoter to the various fields of medicines, foods and drinks, and cosmetics.

Also, this invention allows for the additional effect of promoting the BMAL1 gene-expression to promote muscle-building action. In other words, the non-patent Document 1 (Chatterjee et al.) discloses that the clock gene, i.e. the BMAL1 gene, is an essential gene for forming muscle fibers of the skeletal muscles. Thus, deficiency in BMAL1 decreases muscle mass, and, contrarily, if the BMAL1 is forcibly expressed, a myoblast differentiation is enhanced, thus making it possible to increase muscle mass. Furthermore, it can be expected that thicker muscle fibers increase, and that thin muscle fibers decrease.

As such, an increase in muscle mass by improving the life rhythm makes it possible for increasing fat consumption. Then, anti-obesity and a dietary effect can be expected, thus helping in the prevention of such a metabolic syndrome.

### BRIEF DESCRIPTION OF THE DRAWINGS

FIG. 1 is an HPLC chromatogram of the Kenyan purple-tea extract (purple-tea extract) of the embodiment.

FIG. 2 is a graph showing the relationship between the concentration of the Kenyan purple-tea extract (purple-tea extract) of the embodiment and of the GHG and expression-level of the BMAL1 gene.

FIG. 3 is a schematic diagram showing the periodic change in the expression-level of the clock gene (the BMAL1 gene and the Per gene).

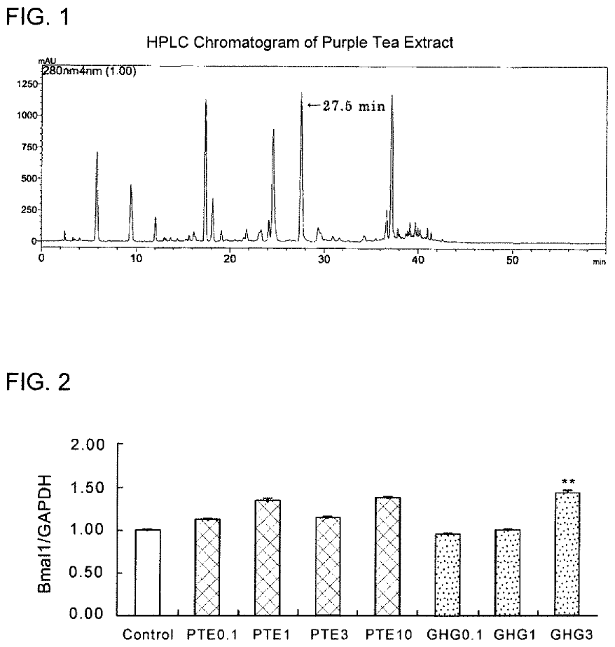

### MODES FOR CARRYING OUT THE INVENTION

Hereinafter, examples of this invention are described.

The circadian-rhythm regulator of this invention is used for regulating the circadian rhythm by the BMAL1 gene-expression-promoting activity. The Brain-Muscle Arnt-Like Protein 1 (BMAL1) is one of the clock genes, which determines the function of the circadian rhythm. It is also called Aryl-hydrocarbon Receptor-Nuclear Translocator-Like (ARNTL).

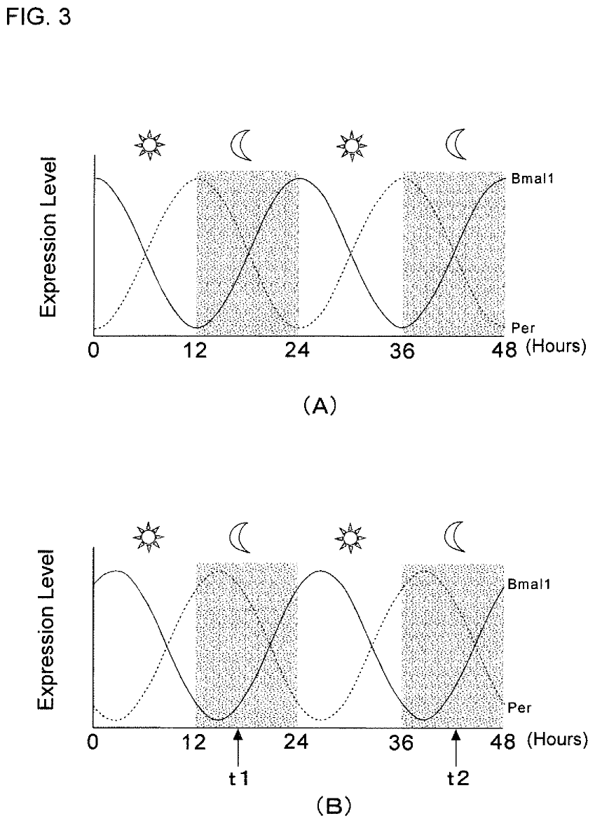

For example, as shown in FIG. 3 (A), in the case that the biological clock is beat out in the circadian rhythm of a normal 24-hour cycle, the BMAL1 gene and the Per gene alternately increase and decrease every 12 hours. That is, the expression-level of the BAML1 gene reaches peak at night, and centrally, the expression-level of the Per gene becomes minimum at night.

In the case that the circadian rhythm functions abnormally due to time differences or the like, phase, frequency and amplitude of the circadian rhythm gets out of normal position. For example, as shown in FIG. 3 (B), in the case that the phase of the circadian rhythm is out of normal position, the circadian-rhythm regulator is applied during the time zone t1 and t2, as the expression-level of the BMAL1 gene increases. Then, the increase-rate of the BMAL1 gene-expression level becomes faster, thus making it possible to progress the phase of the circadian rhythm and to bring it to the normal level.

As such, controlling the expression-level of the BMAL1 gene at the appropriate timing of the circadian rhythm makes it possible to regulate the phase, frequency and amplitude of the circadian rhythm.

The active substance GHG, which promotes the BMAL1 gene-expression level, is represented as the following Chemical Formula 1.

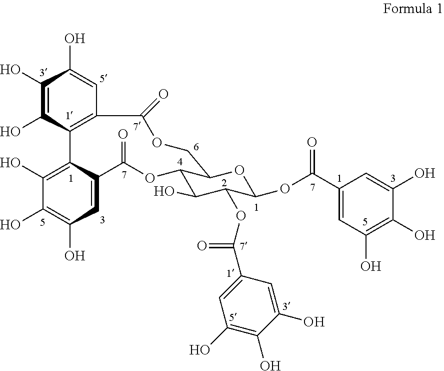

The GHG-content extract of this invention is an extract obtained from Kenyan purple tea and has a GHG of 3 to 99 percent by mass. The form of such GHG can be liquid, solid, semisolid or gel or the like. The solid-content-equivalent quantity of such GHG should be 3 to 99 percent by mass. However, in the case that such GHG-content extract is used as an ingredient of foods and drinks and cosmetics, it should be 3 to 30 percent by mass or preferably 3 to 10 percent by mass. The GHG-content extract of 3 to 10 percent by mass can be obtained efficiently by the following producing method.

The Kenyan purple tea used in obtaining the GHG-content extract is a crossbred-tea plant (scientific name: thea sinensis) that was developed by the Kenyan government. Its variety name is TRFK306. The leaf of Kenyan purple tea contains anthocyanin, is purple, and is also called just “purple tea.” Another purple tea, other than TRFK306, is Sun Rogue or the like that was developed by an agricultural research organization called the National Agriculture and Food Research Organization. Kenyan purple tea contains the specific component of GHG in high concentration that does not exist in other purple teas.

The part of the Kenyan purple tea used in obtaining the GHG-content extract of this invention is not specified. The leaf, stem, root, flower or seed or the like can be used. The leaf is preferable, since it is possible to get from it a high level of GHG.

The GHG-content extract of this invention preferably can be obtained by crushing e.g. the fresh or dried leaf of Kenyan purple tea (hereinafter called purple tea) and then by extracting such fresh or dried leaves by using a polar solvent (with water). Hereinafter the same shall apply. However, the GHG-content extract can be more efficiently extracted by applying a chemical treatment to the purple tea, such as acid or alkaline decomposition or enzyme decomposition or the like.

Specifically, by using the following method, the GHG-content extract can be produced.

Firstly, apply a chemical treatment such as acid or alkaline decomposition or enzyme decomposition or the like to the fresh or dried purple-tea leaves.

Secondly, add the polar solvent to the purple-tea leaves. Then, shake or reflux them by heat to extract the GHG into the solvent.

At this time, not only a polar solvent can be used but also water, alcohol or ketone. Also, a mixture of one or more of these solvents can be used.

It is also preferable to use either aqueous alcohol or aqueous ketone.

An aqueous solvent such as ethanol, methanol, propanol or the like can be used as an aqueous-alcoholic solvent. An aqueous solvent such as acetone, methylethylketone, diethyl ketone, chloroacetone or the like can be used as an aqueous-ketonic solvent. An aqueous-acetone solvent is preferred.

In the case of aqueous ethanol, the ratio of water is 1 to 99.9 percent by mass, preferably 30 to 99 percent by mass, more preferably 40 to 80 percent by mass, most preferably 40 to 60 percent by mass. In the case of aqueous acetone, the amount of acetone preferably is 20 to 99.9 percent by mass, since the GHG can be efficiently extracted within the above range. For the sake of simplicity, an ethanol of 80 percent by mass, with water of 20 percent by mass, should be represented as “80 percent hydrous ethanol.”

In producing the GHG-content extract of this invention, heating and refluxing can be done by the well-known method of using aqueous-alcoholic solvent or aqueous-ketonic solvent. The heat temperature should be 30 to 95 degrees Celsius, preferably 30 to 50 degrees Celsius. The refluxing time should be from one to four hours.

In the process of producing the GHG-content extract of this invention, shaking, stirring or the like can be done accordingly as necessary.

In the process of manufacturing the GHG-content extract of this invention, it is preferable to vacuum and distil the solvent after the GHG extract has been extracted. This process forms a component without using an organic solvent, which process can be applied to a food ingredient to be mixed with foods and drinks such as functional food, healthy food or the like, thus making it possible to meet the safety standards or the like.

In the process of manufacturing the GHG-content extract of this invention, it is possible to extract it in stages by using several different solvents, thus making it possible to produce the GHG-content extract in high concentration.

Specifically, add the purple-tea leaf either to the aqueous-alcoholic solvent or to the aqueous-ketonic solvent. Then, shake such solvent or heat and reflux it to extract the GHG into the solvent, thus obtaining the first extract. Next, by the centrifugal-separation method or the like, separate the extracted GHG from the residue not collected as GHG-content extract and add the other not-chosen-before (aqueous-alcoholic or aqueous-ketonic) solvent to such residue and shake it or heat and reflux it to extract (any remaining) GHG into that solvent, thus obtaining the second extract. Then, mix the first extract with the second extract. Needless to say, the second extract alone can be used as the purple-tea-leaf extract (GHG-content extract).

As the above extractions use several solvents in stages, it is thought that the purple-tea leaf that has gone through the first-extraction process by the aqueous-alcoholic solvent or the aqueous-ketonic solvent would change, so that a feature, i.e. the physical property of the purple-tea leaf would become appropriate for the extraction. Therefore, it can be expected that the second extraction process makes it possible to improve the extraction efficiency even if solvents other than aqueous-alcoholic solvents or aqueous-ketonic solvents are used.

The extracted liquid obtained by the above method can be directly concentrated into the GHG-content extract. Also, such liquid can be freeze-dried and spray-dried into a powder, thus obtaining a powdery GHG-content extract. However, such GHG-content extract is not limited to being in that condition.

Insoluble matter contained in the extract can be removed accordingly by filtering such extract or the like, or such insoluble matter can be crushed into microscopic particles.

As a method for producing the GHG of this invention, it is preferable to fractionate and distillate the GHG-content extract that was obtained by the above process, based on the index that is the already-known GHG, by using an ion-exchange, a size-exclusion chromatography, a high-performance liquid chromatography (HPLC), a gel-filtration or a membrane-separation or the like. Of course, it is possible to extract and distillate the GHG from materials other than Kenyan purple tea. Also, it is possible accordingly to apply an organic-synthetic method.

The circadian-rhythm regulator and BMAL1 gene-expression promoter of this invention can be used as drugs and medicines and quasi-drugs such as tablets, granules, powdered medicines, liquids, powders, capsules, jellies or the like to be shaped by adding base materials and carriers to the GHG or to the GHG-content extract as the active ingredient.

Also, the GHG or GHG-content extract, as the active ingredient of this invention, can be used as the material of various food components, drink components and cosmetic components. These components mean the materials obtained technically by mixing the ingredients appropriate for various uses. These components do not mean any animal or plant materials.

The examples of this invention, regarding foods and drinks, include e.g. edible oils (salad oils), confectionary (chewing gums, candies, caramels, chocolates, cookies, snacks, jellies, gummies, tablet shaped sweets or the like), noodles (Japanese buckwheat noodles called Soba, Japanese wheat noodles called Udon, Japanese noodles called Ramen or the like), dairy food (milk, ice cream, yogurt, or the like), seasoning (fermented rice, barley, soybean paste or the like called Miso, Soy sauce called Shoyu, or the like), soups, drinks (juice, coffee, black tea, green tea, carbonated drink, sports supplement drinks or the like) including general foods and healthy food (tablet type, capsule type or the like), nutritional supplements (nutritious supplement drink or the like). The GHG or GHG-content extract of this invention can be added accordingly to the above foods and drinks.

According to the type of food-and-drink components, the following ingredients can be added: Glucose, fructose, sucrose, maltose, sorbitol, stevioside, corn syrup, lactose, citric acid, tartaric acid, malic acid, succinic acid, lactic acid, L-ascorbic acid, dl-α-tocopherol, sodium erythorbate, glycerin, propylene glycol, glycerin fatty acid ester, polyglycerol fatty acid ester, sucrose fatty acid ester, sorbitan fatty acid ester, propylene glycol fatty acid ester, Gum arabic, carrageenan, casein, gelatin, pectin, agar-agar (gelatin made from seaweed), vitamin B family, nicotinic-acid amide, pantothenate acid calcium, amino acids, calcium salts, pigment, aroma chemicals, preservatives, or the like.

Also, regarding the food-and-drink components having a health maintenance function, the following components of other antioxidants, health-food ingredients or the like can be mixed; e.g. antioxidant, reduced ascorbic acid (i.e. vitamin C), vitamin E, reduced glutacin, tocotrienol, vitamin A derivative, lycopene, rutin, astaxanthin, zeaxanthin, fucoxanthin, uric acid, ubiquinone, coenzyme Q-10, folic acid, garlic extract, allicin, sesamin, lignans, catechin, isoflavone, chalcone, tannins, flavonoids, coumarin, isocoumarines, blueberry extract, arbutin, tannin, anthocyanin, apple polyphenol, grape seed extract, ellagic acid, kojic acid, healthy food ingredient containing serge extract, vitamin A (i.e. V.A), V.B1, V.B2, V.B6, V.B12, V.C, V.D, V.E, V.P, choline, niacin, pantothenic acid, calcium folic acid, EPA, oligosaccharide, dietary fiber, squalene, soybean lecithin, taurine, dunalliela, protein, octacosanol, DHA, egg-yolk lecithin, linoleic acid, lactoferrin, magnesium, zinc, chrome, selenium, kalium, hem iron, oyster extract, chitosan, chitin oligosaccharides, collagen, chondroitin, turmeric, sweetroot, extract of Chinese wolfberry fruit called kukoshi, cinnamon, hawthorn (may), ginger, bracket fungus, shijimi clam (corbicula japonica) extract, snapping turtle, Chinese plantain, chamomilla, chamomile, dandelion, hibiscus, honey, pollen, royal jelly, lime, lavender, rose hip, rosemary, sage, bifidobacteria, streptococcus faecalis, lactobacillus, wheat germ oil, sesame oil, perilla oil, soybean oil, medium chain fatty acid, agaricus, ginko biloba extract, chondroitin, brown rice germ oil, leechee, onion, DHA, EPA, DPA, rubus suavissimus s.lee, plant worm (cordyceps sineusis saccardo), garlic, larvae of a bee, papaya, pu-erh-tea, propolis, Acer nikoense, hericium erinaceum, royal jelly, saw palmetto, hyaluronic acid, collagen, gaba, harp seal oil, shark cartilage, glucosamine, lecithin, phosphatydyl serine, panax notoginseng, mulberry leaf, soybean extract, Echinacea purpurea, acanthopanax senticosus, barley extract, olive leaf, olive, gymnema, banaba, salacia reticulata, garcinia, chitosan, saint john's wort, jujube, carrot, passion flower, broccoli, placenta, coix lacryma bobi. Var. ma-yuen, grape seed, peanut skin, bilberry, black cohosh, milk thistle (silybum marianum), laurel, sage, rosemary, apocynum venetum, black vinegar, bitter gourd, maca, carthamus tinctorius (safflower), linseed, oolong tea, flower aculeus, caffeine, capsaicin, xylo-oligosaccharide, glucosamine, buckwheat, citrus, dietary fiber, protein, prune, spirulina, young green barley leaf, nucleic acid, natural yeast, shiitake mushroom (lentinus edodes), Japanese plum, amino acid, extract of deep sea shark, morinda citrifolia, oyster meat, snapping turtle, champinion, common plantain, acerola, pineapple, banana, peach, apricot, melon, strawberry, raspberry, orange, fucoidan, acer nikoense, cranberry, chondroitin sulfate, zinc, iron, ceramide, silk peptide, glycine, niacin, chaste tree, ceramide, L-cysteine, red wine leaf, millet, horsetail, bition, centrlla asiatica, lonicera caerulea, pycnogenol, petasites japonicus, rhubarb, clove, rosemary, catechin, pu-erh, citric acid, beer yeast, mellilot, black ginger, ginger, curcuma zedoaria, nattokinase, ang-khak (Chinese red rice), tocotrienol, lactoferrin, cinnamon, tartary buckwheat, cocoa, citrus junos (yuzu) seed extract, perilla seed extract, litchi seed extract, evening primrose extract, black rice extract, α-lipoic acid, gaba, green coffee bean extract, Japanese butterbur extract, kiwi fruit seed extract, citrus unshiu (Japanese orange—mikan) extract, red ginger extract, astaxanthin, or the like.

As a more specific method, spray-dry or freeze-dry the active ingredient of this invention i.e. the GHG or GHG-content extract. Especially, in the case of the GHG-content extract, spray-dry or freeze-dry it with a dextrin powder, thus making it into a powder, a granule, a tablet or liquid to mix it easily with foods (instant food or the like). Also, it is possible accordingly to mix it with a binder such as gum arabic or the like to make it into a powder or a granule, thus making it possible to add it to a solid food.

In the case that the circadian-rhythm regulator and the BMAL1 gene-expression promoter of this invention are formed into a pharmaceutical (including medicines and quasi-drugs), the active ingredients (the GHG or GHG-content extract) of this invention can be appropriately mixed with raw materials to form the above pharmaceutical, including e.g. vehicles (glucose, lactose, sucrose, sodium chloride, starch, calcium carbonate, kaolin, crystalline cellulose, cacao oil, hydrogenated vegetable oil, talc, or the like), binders (distilled water, normal saline solution, ethanolic solution, simple syrup, dextrose in water, starch solution, gelatin solution, carboxymethyl cellulose, potassium phosphate, polyvinyl pyrrolidone, or the like), disintegrating agents (alginate sodium, agar-agar, sodium hydrogen carbonate, calcium carbonate, sodium lauryl sulphate, stearic acid monoglyceride, starch, lactose, powdered aracia, gelatin, ethanol, or the like), disintegration-suppressive agents (sucrose, stearin, cacao oil, hydrogenated oil, or the like), absorption promoters (quaternary ammonium base, sodium lauryl sulphate, or the like), adsorbents (glycerin, starch, lactose, kaolin, bentonite, silica acid, or the like), lubricant agents (purified talc, stearate, polyethyleneglycol, polyethylene glycol or the like)

As an administration-method of the above medicines, it is possible to administer them orally in the form of tablets, pills, soft or hard capsules, subtle granules, powders, granules or the like. Also, water-soluble preparations can be orally administered in liquid form. However, such medicines can also be parenterally administered after dispersing such medicine components in a solubilizer such as ethanol, water or the like in the different forms of a medical skin-patch, a lotion, an ointment, a tincture, a cream or the like. Also, the water-soluble preparation of the compounds can be mixed with a dispersant, a suspension agent or a stabilizer or the like, thus making it possible to use such preparation in the form of a medical skin-patch, a lotion, an ointment, a tincture, a cream or the like.

The applied dose can be adjusted according to the method of administration, or to the condition of the disease, or to the age of the patient or the like. However, adults normally can take approx. 5.0 to 200 mg of an active ingredient per day, while children can take 0.5 to 100 mg per day.

The blending-ratio of the active ingredient (i.e. the GHG or GHG-content extract) of this invention can be adjusted according to the form of medicine administered. When such active ingredient is orally or mucosally administered, the applied dose preferably should be about 0.01 to 10.0 wt %. When it is parenterally administered, the dose preferably should be 0.01 to 20 wt %. The dose varies depending on the condition of the patient, so that a dose less than the above amount may be sufficient, or a greater amount may sometimes be needed. Medical components possibly contain other ingredients such as the already-known ingredients regularly used in the pharmaceutical field and those ingredients necessary to make the active ingredient into any appropriate form to be orally applied, which includes e.g. lactose, starch, hydroxypropylcellulose, kaolin, talc, calcium carbonate or the like.

Forms of the cosmetic-components of this invention include e.g. emulsions, soaps, facial cleansers, bath agents, creams, emulsions, skin lotions, colognes, shaving creams, shaving lotion, beauty oils, tanning lotions, sunscreen lotions, face powders, foundations, perfumes, facial masks, nail creams, nail enamels, nail-polish removers, eyebrow pencils, blushers, eye creams, eye shadows, mascaras, eye liners, sticks, lip creams, shampoos, hair conditioners, hair colors, dispersion liquids, cleansing preparations, or the like.

Within the functional range of the active ingredients (GHG or GHG-content extract) of this invention, the above items for external-skin use can be mixed with the ingredients of cosmetics, quasi-drugs, or the like. Those ingredients include, for example, oil, higher alcohol, fatty acids, ultraviolet absorbers, powders, pigments, surface active agents, polyhydric alcohol and sugar, polymers, biologically active ingredients, solvents, antioxidants, aroma chemicals, antiseptics. However, those ingredients usable in the present invention are not limited to these examples.

(1) Specific Examples of Oil

Ester-type oil phase ingredient includes: Triglyceryl 2-ethylhexanoate, cetyl 2-ethylhexanoate, isopropyl myristate, butyl myristate, isopropyl palmitate, ethyl stearate, octyl palmitate, isocetyl isostearate, butyl stearate, butyl myristate, ethyl linoleate, isopropyl linoleate, ethyl oleate, isocetyl myristate, isostearyl myristate, isostearyl palmitate, octyldodecyl myristate, isocetyl isostearate, diethyl sebacate, diisopropyl adipate, isoarachyl neopentanoate, caprylic-capric acid triglyceride, trimethylolpropane tri-2-ethylhexanoate, trimethylolpropane triisostearate, pentaerythritol tetra-2-ethylhexanoate, cetyl caprylate, decyl laurate, hexyl laurate, decyl myristate, myristyl myristate, cetyl myristate, stearyl stearate, decyl oleate, cetyl ricinoleate, isostearyl laurate, isotridecyl myristate, isocetyl myristate, isostearyl myristate, isocetyl palmitate, isostearyl palmitate, octyl stearate, isocetyl stearate, isodecyl oleate, octyldodecyl oleate, octyldodecyl linoleate, isopropyl isostearate, cetostearyl 2-ethylhexanoate, stearyl 2-ethylhexanoate, hexyl isostearate, ethylene glycol dioctanoate, ethylene glycol dioleate, propylene glycol dicaprate, propylene glycol di (caprylate/caprate), propylene glycol dicaprylate, neopentyl glycol dicaprate, neopentyl glycol dioctanoate, glyceryl tricaprylate, glyceryl triundecylate, glyceryl triisopalmitate, glyceryl triisostearate, octyldodecyl neopentanoate, isostearyl octanoate, octyl isononanoate, hexyldecyl neodecanoate, octyldodecyl neodecanoate, isocetyl isostearate, isostearyl isostearate, octyldecyl isostearate, polyglycerin oleate, polyglycerin isostearate, dipropyl carbonate, dialkyl carbonate (C12-18), triisocetyl citrate, triisoarachyl citrate, triisooctyl citrate, lauryl lactate, myristyl lactate, cetyl lactate, octyldecyl lactate, triethyl citrate, acetyltriethyl citrate, acet-yltributyl citrate, trioctyl citrate, diisostearyl malate, 2-ethylhexyl hydroxystearate, 2-ethylhexyl succinate, diisobutyl adipate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoylhydroxystearate, stearyl 12-stearoylhydroxystearate, isostearyl 12-stearoylhydroxystearate or the like.

Hydrocarbon-type oil phase ingredient includes: Squalane, liquid paraffin, α-olefin oligomer, isoparaffin, ceresin, paraffin, liquid isoparaffin, polybutene, microcrystalline wax, Vaseline or the like.

Animal and plant oil, hardened oil thereof, and wax of natural origin:

Animal oils and hardened oils thereof, such as beef tallow, hardened beef tallow, lard, hardened lard, horse oil, hardened horse oil, mink oil, orange roughy oil, fish oil, hardened fish oil, egg yolk oil or the like; plant oils and hardened oils thereof such as avocado oil, almond oil, olive oil, cacao oil, kiwifruit seed oil, apricot kernel oil, kukui nut oil, sesame oil, wheat germ oil, rice germ oil, rice bran oil, safflower oil, shea butter, soybean oil, evening primrose oil, perilla oil, tea seed oil, tsubaki oil (camellia japonica oil), corn oil, rapeseed oil, hardened rapeseed oil, palm kernel oil, hardened palm kernel oil, palm oil, hardened palm oil, peanut oil, hardened peanut oil, castor oil, hydrogenated castor oil, sunflower oil, grape seed oil, jojoba oil, hardened jojoba oil, macadamia nut oil, meadowfoam seed oil, cottonseed oil, hardened cottonseed oil, coconut oil, hardened coconut oil, or the like; and waxes such as beeswax, high acid number beeswax, lanolin, reduced lanolin, hardened lanolin, liquid lanolin, carnauba wax and montan wax, or the like.

Silicone-Type Oil Phase Ingredient:

Dimethylpolysiloxane, methylphenylpolysiloxane, methylcyclopolysiloxane, octamethylpolysiloxane, decamethylpolysiloxane, dodecamethylcyclosiloxane, methylhydrogenpolysiloxane, polyether-modified organopolysiloxane, dimethylsiloxanemethylcetyloxysiloxane copolymer, dimethylsiloxane-methylstearoxysiloxane copolymer, alkyl-modified organopolysiloxane, terminal-modified organopolysiloxane, amino-modified silicone oil, amino-modified organopolysiloxane, dimethiconol, silicone gel, acryl silicone, trimethylsiloxysilicic acid, silicone RTV rubber or the like.

Fluorine-Type Oil Phase Ingredient:

Perfluoropolyether, fluorine-modified organopolysiloxane, fluorinated pitch, fluorocarbon, fluoroalcohol, fluoroalkyl-polyoxyalkylene-comodified organopolysiloxane, or the like.

(2) Specific Examples of Higher Alcohol

Lauryl alcohol, myristyl alcohol, cetyl alcohol, stearyl alcohol, isostearyl alcohol, oleyl alcohol, behenyl alcohol, 2-ethylhexanol, hexadecyl alcohol, octyl dodecanol or the like.

(3) Specific Examples of Fatty Acid

Caprylic acid, capric acid, undecylenic acid, lauric acid, myristic acid, palmitic acid, palmitoleic acid, stearic acid, isostearic acid, oleic acid, linoleic acid, linolenic acid, arachic acid, arachidonic acid, behenic acid, erucic acid, 2-ethylhexanoic acid or the like.

(4) Specific Examples of Ultraviolet Absorber

Para-aminobenzoic acid, amyl para-aminobenzoate, ethyldihydroxypropyl para-aminobenzoate, glyceryl para-aminobenzoate, ethyl para-aminobenzoate, octyl para-aminobenzoate, octyldimethyl para-aminobenzoate, ethylene glycol salicylate, octyl salicylate, triethanolamine salicylate, phenyl salicylate, butylphenyl salicylate, benzyl salicylate, homomenthyl salicylate, benzyl cinnamate, octyl para-methoxycinnamate, 2-ethylhexyl para-methoxycinnamate, glyceryl mono-2-ethyl hexanoate di-para-methoxycinnamate, isopropyl para-methoxycinnamate, diethanolamine para-methoxyhydrocinnamate, diisopropyl diisopropylcinnamic acid ester mixture, urocanic acid, ethyl urocanate, hydroxymethoxybenzophenone, hydroxymethoxybenzophenone sulfonic acid and a salt thereof, dihydroxymethoxybenzophenone, sodium dihydroxymethoxybenzophenonedisulfonate, dihydroxybenzophenone, dihydroxydimethoxybenzophenone, hydroxyoctoxybenzophenone, tetrahydroxybenzophenone, butylmethoxydibenzoylmethane, 2,4, 6-trianilino-p-(carbo-2-ethylhexyl-1-oxy)-1, 3,5-triazine, 2-(2-hydroxy-5-methylphenyl) benzotriazole, methyl-0-aminobenzoate, 2-ethylhexyl-2-cyano-3,3-diphenylacrylate, phenylbenzimidazole sulfuric acid, 3-(4-methylbenzylidene) camphor, isopropyldibenzoylmethane, 4-(3, 4-dimethoxyphenylmethylene)-2, 5-doxy-1-imidazolidinepropionate, and polymer derivatives and silane derivatives thereof, or the like.

(5) Specific Examples of Powder and Pigment

Pigments such as Food Red 104, Food Red 201, Food Yellow 4, Food Blue 1 and Food Black 401; lake pigments such as Food Yellow 4 AL lake and Food Yellow 203 BA lake; polymers such as nylon powder, silk powder, urethane powder, Teflon® powder, silicone powder, polymethyl methacrylate powder, cellulose powder, starch, silicone elastomer spherical powder and polyethylene powder; color pigments such as yellow iron oxide, red iron oxide, black iron oxide, chromium oxide, carbon black, ultramarine and iron blue; white pigments such as zinc oxide, titanium oxide and cerium oxide; extender pigments such as talc, mica, sericite, kaolin and plate barium sulfate; pearl pigments such as mica titanium; metal salts such as barium sulfate, calcium carbonate, magnesium carbonate, aluminum silicate and magnesium silicate; inorganic powders such as silica and alumina; metal soaps such as aluminum stearate, magnesium stearate, zinc palmitate, zinc myristate, magnesium myristate, zinc laurate and zinc undecylenate; bentonite; smectite; and boron nitride. From among the above ingredients, one only can be used, or two or more can be used together.

The shape (e.g., sphere, bar, needle, plate, amorphous, scale, spindle) and the particle size of these powders are not particularly limited. These powders may or may not be previously surface-treated by a conventionally known surface treatment such as fluorine compound treatment, silicone treatment, silicone resin treatment, pendant treatment, saline coupling agent treatment, titanium coupling agent treatment, lubricant treatment, N-acylated lysine treatment, polyacrylic acid treatment, metal soap treatment, amino acid treatment, lecithin treatment, inorganic compound treatment, plasma treatment and mechanochemical treatment.

(6) Specific Examples of Surfactant

Anionic Surfactant:

Fatty acid soap, a-acyl sulfonate, alkyl sulfonate, alkylallyl sulfonate, alkylnaphthalene sulfonate, alkyl sulfate, POE alkyl ether sulfate, alkylamide sulfate, alkyl phosphate, POE alkyl phosphate, alkylamide phosphate, alkyloylalkyl taurine salt, N-acylamino acid salt, POE alkyl ether carbonate, alkyl sulfosuccinate, sodium alkylsulfoacetate, acylated hydrolyzed collagen peptide salt, perfluoroalkylphosphoric acid ester or the like.

Cationic Surfactant:

Alkyltrimethylammonium chloride, stearyltrimethylammonium chloride, stearyltrimethylammonium bromide, cetostearyltrimethylammonium chloride, distearyldimethylammonium chloride, stearyldimethylbenzylammonium chloride, behenyltrimethylammonium bromide, benzalkonium chloride, behenic acid amidopropyldimethyl hydroxypropylammonium chloride, diethylaminoethylamide stearate, dimethylaminoethylamide stearate, dimethylaminopropylamide stearate, lanolin derivative quaternary ammonium salt or the like.

Amphoteric Surfactant:

Carboxybetaine type, amidobetaine type, sulfobetaine type, hydroxysulfobetaine type, amidosulfobetaine type, phosphobetaine type, aminocarboxylate type, imidazoline derivative type, amidoamine type or the like.

Nonionic Surfactant:

Propylene glycol fatty acid ester, glycerin fatty acid ester, polyglycerin fatty acid ester, sorbitan fatty acid ester, POE sorbitan fatty acid ester, POE sorbitol fatty acid ester, POE glycerin fatty acid ester, POE alkyl ether, POE fatty acid ester, POE hydrogenated castor oil, POE castor oil, POE-POP copolymer, POE-POP alkyl ether, polyether-modified silicone lauric acid alkanolamide, alkylamine oxide, hydrogenated soybean phospholipid or the like.

Natural-Type Surfactant:

Lecithin, saponin, sugar-type surfactant or the like.

(7) Specific Examples of Polyhydric Alcohol and Sugar

Ethylene glycol, diethylene glycol, polyethylene glycol, propylene glycol, dipropylene glycol, polypropylene glycol, glycerin, diglycerin, polyglycerin, 3-methyl-1, 3-butanediol, 1,3-butylene glycol, sorbitol, mannitol, raffinose, erythritol, glucose, sucrose, fruit sugar, xylitol, lactose, maltose, maltitol, trehalose, alkylated trehalose, mixed isomerized sugar, sulfated trehalose, pullulan or the like. Chemically modified products thereof can also be used.

(8) Specific Examples of Polymer Compound

Anionic polymer compounds such as acrylic acid ester/methacrylic acid ester copolymer (PLUS-SIZE, produced by Sogokagaku K. K.), vinyl acetate/crotonic acid copolymer (Resin 28-1310, produced by NSC), vinyl acetate/crotonic acid/vinyl neodecanate copolymer (28-2930, produced by NSC), methyl vinyl ether maleic acid half ester (GANTREZ ES, produced by ISP), T-butyl acrylate/ethyl acrylate/methacrylic acid copolymer (RUBIMER, produced by BASF), vinylpyrrolidone/vinyl acetate/vinyl propionate copolymer (RUBISCOL VAP, produced by BASF), vinyl acetate/crotonic acid copolymer (RUBISET CA, produced by BASF), vinyl acetate/crotonic acid/vinylpyrrolidone copolymer (RUBISET CAP, produced by BASF), vinylpyrrolidone/acrylate copolymer (RUBIFLEX, produced by BASF), acrylate/acrylamide copolymer (ULTRAHOLD, produced by BASF), vinyl acetate/butyl maleate-isobornyl acrylate copolymer (ADVANTAGE, produced by ISP), carboxy vinyl polymer (CARBOPOL, produced by BF Goodrich) and acrylic acid-alkyl methacrylate copolymer (PAMUREN, produced by BF Goodrich); amphoteric polymer compounds such as acetic acid amphoteric compound of dialkylaminoethyl methacrylate polymer (YUKAFORMER, produced by Mitsubishi Chemical) and octylacrylamide acrylate/hydroxypropyl acrylate/butylaminoethyl methacrylate copolymer (AMPHOMER, produced by NSC); cationic polymer compounds such as quaternized compound of vinylpyrrolidone/dimethylaminoethyl methacrylate (GAFQUAT, produced by ISP) and methyl vinyl imidazolium chloride/vinylpyrrolidone copolymer (RUBICOTE, produced by BASF); and nonionic polymer compounds such as polyvinylpyrrolidone/vinyl acetate copolymer (RUBISCOL VA, produced by BASF) and vinylpyrrolidone/dimethylaminoethyl methacrylate copolymer (COPOLYMER VC713, produced by ISP).

In addition, polymer compounds of natural origin, such as cellulose and derivatives thereof, calcium alginate, pullulan, agar, gelatin, tamarind seed polysaccharides, xanthane gum, carrageenan, high-methoxyl pectin, low-methoxyl pectin, guar gum, gum arabic, crystal cellulose, arabino galactan, karaya gum, tragacanth gum, alginic acid, albumin, casein, cardrun, gellan gum, dextran or the like, can also be suitably used.

(9) Specific Examples of Biologically Active Ingredient

The biologically active ingredient may include substances which are capable of imparting some biological activity to skin, when such a substance is applied to the skin. Specific examples thereof may include: whitening ingredient, immunomodulator, age resistor, ultraviolet protection, slimming agent, skin tightening agent, antioxidant, hair restorer, hair growing agent, moisturizer, blood circulation accelerator, antibacterial agent, bactericide, desiccant, cooling agent, warming agent, vitamin compound, amino acid, wound healing accelerator, torpent, analgetic, cell activator and enzyme ingredient.

Suitable examples of the ingredient to be blended therefor may include: angelica extract, avocado extract, hydrangea extract, althea extract, arnica extract, aloe extract, apricot extract, apricot core extract, ginkgo extract, fennel extract, turmeric extract, oolong tea extract, rose fruit extract, echinacea leaf extract, scutellaria root extract, phellodendron bark extract, goldthread extract, barley extract, hypericum extract, white nettle extract, watercress extract, orange extract, sea salt, seaweed extract, hydrolyzed elastin, hydrolyzed wheat powder, hydrolyzed silk, chamomile extract, carrot extract, artemisia capillaris extract, glycyrrhiza extract, Hibiscus sabdariffa extract, pyracantha fortuneana fruit extract, cinchona extract, cucumber extract, guanosine, gardenia extract, sasa albo-marginata extract, sophora root extract, walnut extract, grapefruit extract, clematis extract, chlorella extract, mulberry bark extract, gentian extract, black tea extract, yeast extract, burdock extract, fermented rice bran extract, rice germ oil, comfrey extract, collagen, cowberry extract, asiasarum root extract, bupleurum falcatum root extract, umbilical cord extract, salvia extract, saponaria extract, bamboo grass extract, crataegus extract, zanthoxylum fruit extract, shiitake mushroom extract, rehmannia root extract, lithospermum root extract, perilla extract, linden extract, filipendula extract, peony root extract, calamus rhizome extract, birch extract, horsetail extract, ivy extract, hawthorn extract, sambucus nigra extract, yarrow extract, peppermint extract, sage extract, mallow extract, cnidium rhizome extract, swertia herb extract, soy extract, jujube extract, wild thyme extract, green tea extract, clove extract, cogon extract, citrus unshiu peel extract, angelica root extract, calendula extract, peach seed extract, bitter orange extract, houttuynia extract, tomato extract, natto extract, ginseng extract, garlic extract, wild rose extract, hibiscus sabdariffa flower extract, ophiopogon tuber extract, parsley extract, honey, witch hazel extract, pellitory extract, isodonis extract, matricaria extract, loquat extract, coltsfoot extract, butterbur scape extract, Poria cocos extract, butcher bloom extract, grape extract, propolis, luffa extract, safflower extract, peppermint extract, linden extract, peony extract, hop extract, pine extract, horse chestnut extract, skunk cabbage extract, sapindaceae extract, balm mint extract, peach extract, cornflower extract, eucalyptus extract, saxifrage extract, coix seed extract, mugwort extract, lavender extract, apple extract, lettuce extract, lemon extract, Chinese milk vetch extract, rose extract, rosemary extract, Roman chamomile extract, royal jelly extract or the like.

Other examples may include biopolymers such as deoxyribonucleic acid, mucopolysaccharide, sodium hyaluronate, sodium, elastin, chitin, chitosan and hydrolyzed eggshell membrane; moisture retentive ingredients such as amino acid, hydrolyzed peptide, sodium lactate, urea, sodium pyrrolidonecarboxylate, betaine, whey and trimethylglycine; oily ingredients such as sphingolipid, ceramide, phytosphingosine, cholesterol, cholesterol derivatives and phospholipid; anti-inflammatory such as E-aminocaproic acid, glycyrrhizic acid, -glycyrrhetic acid, lysozyme chloride, guaiazlene and hydrocortisone; vitamins such as vitamin A, vitamin B2, vitamin B6, vitamin D, vitamin E, calcium pantothenate, biotin and nicotinic acid amide; active ingredients such as allantoin, diisopropylamine dichloroacetate and 4-aminomethylcyclohexanecarboxylic acid; antioxidants such as tocopherol, carotenoid, flavonoid, tannin, lignin and saponin; cell activators such as a-hydroxy acid and hydroxy acid; blood circulation accelerators such as y-orizanol and vitamin E derivatives; wound healing agents such as retinol and retinol derivatives; whitening agents such as albumin, kojic acid, placenta extract, sulfur, ellagic acid, linoleic acid, tranexamic acid and glutathione; and hair growing agents such as cepharanthine, glycyrrhiza extract, capsicum tincture, hinokitiol, iodized garlic extract, pyridoxine hydrochloride, DL-a-tocopherol, DL-a-tocopheryl acetate, nicotinic acid, nicotinic acid derivatives, calcium pantothenate, D-pantothenyl alcohol, acetyl pantothenylethyl ether, biotin, allantoin, isopropylmethylphenol, estradiol, ethynyl estradiol, capronium chloride, benzalkonium chloride, diphenhydramine hydrochloride, Takanal, camphor, salicylic acid, vanillylamide nonylate, vanillylamide nonanoate, pyroctone olamine, glyceryl pentadecanoate, L-menthol, mononitroguaiacol, resorcinol, y-aminobutyric acid, benzethonium chloride, mexiletine hydrochloride, auxin, female hormone, cantharis tincture, cyclosporine, zinc pyrithione, hydrocortisone, minoxidil, polyoxyethylene sorbitan monostearate, peppermint oil and SASANISHIKI extract, placenta products, Citrus junos seed extract, blueberry extract, lingonberry extract, Cistanche tubulosa extract, black rice extract, green coffee bean extract, resveratrol extract, kiwifruit seed extract, strawberry seed extract, cherry extract, or the like.

(10) Specific Examples of Antioxidant

Sodium hydrogensulfite, sodium sulfite, erythorbic acid, sodium erythorbate, dilauryl thiodipropionate, tocopherol, tolylbiguanide, nordihydroguaiaretic acid, parahydroxy anisole, butylhydroxy anisole, dibutylhydroxy toluene, ascorbyl stearate, ascorbyl palmitate, octyl gallate, propyl gallate, carotenoid, flavonoid, tannin, lignin, saponin and plant extracts having antioxidant effect, such as apple extract, clove extract or the like.

(11) Specific Examples of Solvent

Purified water, ethanol, lower alcohol, ethers, LPG, fluorocarbon, N-methylpyrrolidone, fluoroalcohol, volatile linear silicone, next generation Freon or the like.

### EXAMPLES

Hereinafter, the specific example of this invention is described. The following example is just one of the examples of this invention, and the scope of this invention is not limited to that example.

Also, as one of the examples of this invention, the BMAL1 gene was evaluated regarding rat-muscle myoblasts and identified that the result of such an evaluation of rat-muscle myoblasts can be applied to other organism species and cell species, since the core system of the clock gene is mutually shared with any organism species and cell species.

(Preparation of Purple-Tea Extract (GHG-Content Extract)

50 grams of Kenyan purple-tea leaf is immersed in 500 mL of 50% aqueous solution of ethanol and then stirred and heated and refluxed at 40 degrees Celsius for two hours until such solution becomes the extraction-liquid. 400 mL of such extraction-liquid is obtained by suction-filtration. Then, such extraction-liquid is concentrated and dried into 16.6 grams of the purple-tea extract (GHG-content extract).

Analysis of the component of the purple-tea extract (GHG-content extract) The obtained purple-tea extract (GHG-content extract) was analyzed by using the HPLC method. It was verified that Kenyan purple tea shows the peak of its peculiar component at the point of 27.5 min, which is not contained in ordinary tea plants such as green tea, oolong tea, black tea or the like.

Preparation of Samples

350 mg of the purple-tea extract (GHG-content extract) was dissolved in 30% aqueous-solution of methanol, and the volume was fixed at 20 mL in the measuring flask. The solution was diluted twice and filtered and then analyzed by the HPLC. The condition of the HPLC analysis was as follows. Chart 1 shows the concentration of Mobile phase B proceeding with time.

Condition of the HPLC Analysis
Current speed: 0.7 ml/min
Mobile phase A: 0.3% TFA solution
Mobile phase B: Acetonitrile
Gradient: As shown in Chart 1, below.
Chromatography: TOSOH CORPORATION: TSKGEL ODS-80TS QA(4.6×150 mm)
Chromatography temperature: 35 degrees Celsius
Sample injection dose: 10 μL
Detection wavelength: 280 nm

CHART 1

| Time | Mobile phase B |
|---|---|
| (min) | Concentration (%) |
| 0.0 | 5 |
| 4.0 | 5 |
| 4.5 | 10 |
| 27.0 | 15 |
| 47.0 | 55 |
| 48.0 | 90 |
| 50.0 | 90 |
| 51.0 | 5 |
| 60.0 | 5 |

The above peculiar components were separated and purified, and then an NMR analysis was done. The result is shown in Chart 2, below.

As shown in Chart 2, as a result of the comparison of the NMR analysis-value to the reference-value, the known component 1,2-di-O-galloyl-4,6-O—(S)-hexahydroxydiphenoyl-ß-D-glucose (GHG) was identified

CHART 2
Chart 2. C-NMR Data of GHG
of purple tea component

| Carbon | Purified component | GHG literature |
|---|---|---|
| No. | derived from purple tea | value 1) |
| glucose | | |
| 1 | 94.4 | 94.4 |
| 2 | 74.6 | 74.7 |
| 3 | 74.1 | 74.1 |
| 4 | 73.1 | 73.1 |
| 5 | 73.8 | 73.8 |
| 6 | 64.1 | 64.1 |
| galloyl | | |
| 1 | 119.9 | 119.9 |
| 2.6 | 110.6 | 110.6 |
| 3.5 | 146.5 | 146.5 |
| 4 | 140.6 | 140.7 |
| 7 | 166.3 | 166.4 |
| 1′ | 120.9 | 120.9 |
| 2′.6′ | 110.4 | 110.5 |
| 3′.5′ | 146.4 | 146.4 |
| 4′ | 140.1 | 140.1 |
| 7′ | 167.2 | 167.3 |
| HHDP | | |
| 1 | 116.9 | 116.9 |
| 2 | 126.2 | 126.2 |
| 3 | 108.6 | 108.7 |
| 4 | 145.9 | 145.9 |
| 5 | 137.7 | 137.7 |
| 6 | 144.9 | 144.9 |
| 7 | 169.4 | 169.4 |
| 1′ | 116.6 | 116.6 |
| 2′ | 126.5 | 126.5 |
| 3′ | 108.4 | 108.5 |
| 4′ | 145.8 | 145.8 |
| 5′ | 137.4 | 137.4 |
| 6′ | 144.8 | 144.8 |
| 7′ | 169.8 | 169.9 |

1) Chem. Pharm. Bull. 57(11) 1284-1288 (2009)

The commercially prepared GHG-purified product is set as a standard substance, and the quantitative analysis was done by the HPLC. The result was that the purple-tea extract (GHG-content extract) of Example 1 contains GHG of 8.70 percent by mass.

The purple-tea extract (GHG-content extract) was prepared twice as much more in the same way as described above, and the GHG content in the extract was measured in the same way. The result showed that the GHG-content was 6.79% by mass (of the second preparation) and 6.38% by mass (of third preparation) respectively. Therefore, it was verified that the purple-tea extract (GHG-content extract) prepared according to the method of this invention contained GHG of approximately six to nine percent by mass.

Evaluation of the BMAL1 Gene-Expression-Promotion Activity

Differentiation-induction was done to the rat-muscle myoblasts C2Cl2. At the same time, the purple-tea extract of 0.1, 1.0, 3.0 and 10 μg/mL, and the GHG of 0.1, 1.0 and 3 μg/mL, was respectively added to such myoblasts and cultivated for one week. After that, an RNA extraction was done to determine, by using a reverse-transcribed cDNA, the amount of mRNA and of the BMAL1 gene.

The result is shown in FIG. 2. Therein, the amount of mRNA shows the average±standard deviation (mean±SD). A significance-test was done by the Dunnett Method, and the double asterisk ** means p<0.01. Also, no active components were added to the “control,” but the purple-tea extract (GHG-content extract) was added to the “PTE.”

Regarding the purple-tea extract (GHG-content extract) and the GHG, the expression-level of the BMAL1 against the Control was compared. In the case that the purple-tea extract (GHG-content extract) was added, the expression-level of the BMAL1 gene was seen to have increased in each concentration of 0.1, 1.0 and 10 μg/mL. Also, in the case that the GHG was added, the expression-level significantly was seen to have increased in the concentration of 3 μg/mL. As result, it was verified that the purple-tea extract (GHG-content extract) and the GHG had efficiently promoted the expression level of the BMAL1 gene in the rat-muscle myoblasts C2Cl2.

### Blending Example

Hereinafter, the blending-examples of each ingredient for regulating the circadian rhythm, or for promoting the BMAL1 gene-expression, are described. The purple-tea extract (GHG-content extract) of the blending examples, below, are prepared in similar ways as the above examples. It is also possible to blend the GHG purified-product in addition to the purple-tea extract (GHG-content extract).

### Blending Example 1: Chewing Gums

| | | |
|---|---|---|
| Sugar | 53.0 | wt % |
| Gum base | 20.0 | |
| Glucose | 10.0 | |
| Starch syrup | 16.0 | |
| Aroma chemical | 0.5 | |
| Purple-tea extract (GHG-content extract) | 0.5 | |
| | 100.0 | wt % |

### Blending Example 2: Gummies

| | | |
|---|---|---|
| Reduction sugar | 40.0 | wt % |
| Granulated sugar | 20.0 | |
| Glucose | 20.0 | |
| Gelatin | 4.7 | |
| Water | 9.68 | |
| Grape juice | 4.0 | |
| Grape flavor | 0.6 | |
| Pigment | 0.02 | |
| Purple-tea extract (GHG-content extract) | 1.0 | |
| | 100.0 | wt % |

### Blending Example 3: Candies

| | | |
|---|---|---|
| Sugar | 50.0 | wt % |
| Starch syrup | 33.0 | |
| Water | 14.4 | |
| Organic acid | 2.0 | |
| Aroma chemical | 0.2 | |
| Purple-tea extract (GHG-content extract) | 0.4 | |
| | 100.0 | wt % |

### Blending Example 4: Yogurt (Hard Type/Soft Type)

| | | |
|---|---|---|
| Milk | 41.5 | wt % |
| Powdered skim milk | 5.8 | |
| Sugar | 8.0 | |
| Agar-agar | 0.15 | |
| Gelatin | 0.1 | |
| Lactic-acid bacterium | 0.005 | |
| Purple-tea extract (GHG-content extract) | 0.4 | |

| | |
|---|---|
| Aroma chemical | a minute amount |
| Water | the rest |

| | |
|---|---|
| 100.0 | wt % |

### Blending Example 5: Soft Drinks

| | | |
|---|---|---|
| Fructose-glucose solution | 30.0 | wt % |
| Emulsifying agent | 0.5 | |
| Purple-tea extract (GHG-content extract) | 0.05 | |

| | |
|---|---|
| Aroma chemical | appropriate amount |
| Distilled water | the rest |

| | |
|---|---|
| 100.0 | wt % |

### Blending Example 6: Soft Capsules

| | | |
|---|---|---|
| Grape seed oil | 87.0 | wt % |
| Emulsifying agent | 12.0 | |
| Purple-tea extract (GHG-content extract) | 1.0 | |
| | 100.0 | wt % |

### Blending Example 8: Tablets

| | | |
|---|---|---|
| Lactose | 54.0 | wt % |
| Crystalline cellulose | 30.0 | |
| Starch-splitting product | 10.0 | |
| Glycerin fatty-acid ester | 5.0 | |
| Purple-tea extract (GHG-content extract) | 1.0 | |
| | 100.0 | wt % |

### Blending Example 8: Oral-Granule Medicines (Drugs and Medicines)

| | | |
|---|---|---|
| Lactose | 30.0 | wt % |
| Corn starch | 60.0 | |
| Crystalline cellulose | 8.0 | |
| Polyvinylpyrrolidone | 1.0 | |
| Purple-tea extract (GHG-content extract) | 1.0 | |
| | 100.0 | wt % |

### Blending Example 9: Tablet-Shaped Sweets

| | | |
|---|---|---|
| Sugar | 76.4 | wt % |
| Glucose | 19.0 | |
| Glycerine fatty acid ester | 0.2 | |
| Purple-tea extract (GHG-content extract) | 0.5 | |
| Distilled water | 3.9 | |
| | 100.0 | wt % |

### Blending Example 10: Cosmetic Creams

| Squalene | 20.0 | wt % |
|---|---|---|
| Beeswax | 5.0 | |
| Purified jojoba oil | 5.0 | |
| Glycerin Monostearate | 2.0 | |
| Polyoxyethylene (20) Sorbitan monostearate | 2.0 | |
| Purple-tea extract (GHG-content extract) | 2.0 | |
| Preservatives | appropriate amount | |
| Aroma chemicals | appropriate amount | |
| Distilled water | the rest | |
| | 100.0 | wt % |

### Blending Example 11: Skin Lotions

| | | |
|---|---|---|
| Ethanol | 5.0 | wt % |
| Glycerin | 2.0 | |
| 1,3-butylene glycol | 2.0 | |
| Polyethylene oleyl ether | 0.5 | |
| Sodium citrate | 0.1 | |
| Citric acid | 0.1 | |
| Purple-tea extract (GHG-content extract) | 0.1 | |

| | |
|---|---|
| Distilled water | the rest |

| | |
|---|---|
| 100.0 | wt % |

### Blending Example 12: Body Gels

| Macadamia nut oil | 2.0 | wt % |
|---|---|---|
| Octyl decyl myristate | 10.0 | |
| Methylphenyl polysiloxane | 5.0 | |
| Behenyl alcohol | 3.0 | |
| Stearic acid | 3.0 | |
| Bathyl alcohol | 1.0 | |
| Glycel monostearate | 1.0 | |
| Tetra-oleic acid polyoxyethylene sorbit | 2.0 | |
| Hydrogenated-soybean phosphatide | 1.0 | |
| Ceramide | 0.1 | |
| Retinol palmitate | 0.1 | |
| Preservative agent | appropriate amount | |
| Centella-asiatica extract | 1.0 | |
| Purple-tea extract (GHG-content extract) | 1.0 | |
| 1,3-butylene glycol | 5.0 | |

| | |
|---|---|
| Distilled water | the rest |

| | |
|---|---|
| 100.0 | wt % |

### Blending Example 12: Cosmetic Emulsion

| | | |
|---|---|---|
| Squalene | 4.0 | wt % |
| Vaseline | 2.5 | |
| Cetanol | 2.0 | |
| Glycerin | 2.0 | |
| Oleophilic-glycerine monostearate | 1.0 | |
| Stearic acid | 1.0 | |
| L-arginine | 1.0 | |
| Purple-tea extract (GHG-content extract) | 0.5 | |
| Potassium hydroxide | 0.1 | |

| | |
|---|---|
| Aroma chemical | a minute amount |
| Distilled water | the rest |

| | |
|---|---|
| 100.0 | wt % |

### Blending Example 13: Bath Agent (Liquid Type)

| | | |
|---|---|---|
| Propylene glycol | 50.0 | wt % |
| Ethanol | 20.0 | |
| Sodium sulphate | 5.0 | |
| Purple-tea extract | 0.5 | |
| Lanoline | 0.5 | |
| Avocado oil agent | 0.5 | |
| Pigment | 1.5 | |
| Aroma chemical | 22.0 | |
| | 100.0 | wt % |

### Blending Example 15: Shampoo

| Sodium-polyoxyethylene alkyl-ether sulfate | 15.0 | wt % |
|---|---|---|
| (E. 02 mol) | | |
| Coconut fatty-acid diethanolamide | 5.0 | |
| Glycerin | 3.0 | |
| Purple-tea extract (GHG-content extract) | 0.4 | |
| Ethanol | 5.0 | |
| Aroma chemical and preservative | appropriate amount | |
| Ion-exchanged water | the rest | |
| | 100.0 | wt % |

### Blending Example 16: Hair Cream

| | | |
|---|---|---|
| Liquid paraffin | 20.0 | wt % |
| Solid paraffin | 3.0 | |
| Polyoxyethylene-alkyl ether (E. 015 mol) | 2.0 | |
| Sorbitan sesquioleate | 1.0 | |
| Purple-tea extract (GHG-content extract) | 0.2 | |
| Ethanol | 10.0 | |
| Potassium hydroxide | 0.1 | |
| Glycerin | 3.0 | |

| | |
|---|---|
| Aroma chemical and preservative | appropriate amount |

| | |
|---|---|
| 100.0 | wt % |

### Blending Example 17: Ointment

| | | |
|---|---|---|
| White beeswax | 5.0 | wt % |
| Purified lanolin | 5.0 | |
| Purple-tea extract (GHG-content extract) | 1.0 | |
| Aroma chemical | 0.1 | |

| | |
|---|---|
| Vaseline | the rest |

| | |
|---|---|
| 100.0 | wt % |

### INDUSTRIAL APPLICABILITY

As described above, the circadian-rhythm regulator and the BMAL1 gene-expression promoter of this invention allow for the GHG that is contained in the Kenyan purple tea to regulate the circadian rhythm of living organisms, including man, which cures disorders of physiological activity such as sleep, awakening, hormonal secretion, blood pressure, body temperature or the like, thus making it possible to prevent or to treat lifestyle-related diseases such as sleep disorder or skin disorder, obesity, diabetes, high-blood pressure or the like.

1-10. (canceled)

11. A method of regulating the circadian rhythm of an individual comprising administering an effective amount of a circadian-rhythm regulator containing 1,2-di-O-galloyl-4,6-O—(S)-hexahydroxydiphenoyl-β-D-glucose as the active ingredient to the individual for the purpose of returning the circadian rhythm to a normal level and/or promoting the BMAL1 gene-expression.

12. The method of claim 11, wherein the circadian-rhythm regulator is contained in a food component, medicinal component, cosmetic component or a drink component.

13. The method of claim 11, wherein the circadian-rhythm regulator is an extract derived from Kenyan purple tea.

14. The method of claim 12, wherein the cosmetics component is selected from the group consisting of cosmetic cream, skin lotion, body gel, cosmetic emulsion, liquid bath agent, shampoo, hair cream, facial cleansers, beauty oils, shaving lotions, tanning lotions, face powders, foundations, sunscreens, perfumes, facial masks, soaps, nail enamels, nail polish, eyebrow pencils, blushers, eye creams, eye shadows, mascaras, eye liner, sticks, lip creams, hair conditioners, hair colors, dispersion liquids, and cleansing preparations.

15. The method of claim 12, wherein the food component is selected from the group consisting of chewing gum, gummies, candy, yogurt, cookies, jellies, noodles, ice cream, seasoning, sauces, tablet-shaped sweets, nutritional supplements, oils, caramels, chocolates, and snacks.

16. The method of claim 12, wherein the drink component is selected from the group consisting of soft drinks, soup, coffee, tea, sports drinks, nutritional drinks, juices, and milk.

17. The method of claim 12, wherein the medicinal component is selected from the group consisting of tablets, pills, hard or soft capsules, granules, powders, liquids, skin patches, ointment, tincture, lotion, and cream.

18. The method of claim 11, wherein the circadian-rhythm regulator is administered during a predetermined time period with respect to a circadian-rhythm cycle.

19. The method of claim 13, wherein the extract comprises 3 to 99 wt. % of 1,2-di-O-galloyl-4,6-O—(S)-hexahydroxydiphenoyl-β-D-glucose.

20. the method of claim 12, wherein the food, cosmetic, or drink components comprises 3 to 30 wt. % of an extract containing 1,2-di-O-galloyl-4,6-O—(S)-hexahydroxydiphenoyl-β-D-glucose, the extract containing from 3 to 99 wt. % of the 1,2-di-O-galloyl-4,6-O—(S)-hexahydroxydiphenoyl-β-D-glucose.
